(19) United States
(12) Patent Application Publication
(10) Pub. No.: US 2009/0263469 A1
(43) Pub. Date: Oct. 22, 2009

(54) BIGUANIDE-CONTAINING LIPOSOMES

(75) Inventors: Christian Rohrer, Linz (AT); Tim Axel Wilhelms, Koblenz (DE); Andreas Wagner, Baden (AT)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: LOHMANN & RAUSCHER GMBH & CO. KG, Neuwied (DE)

(21) Appl. No.: 12/295,677

(22) PCT Filed: Mar. 15, 2007
(86) PCT No.: PCT/EP2007/002287

§ 371 Date: Jun. 18, 2009

(30) Foreign Application Priority Data
Apr. 1, 2006 (DE) 10 2006 015 271.9

(51) Int. Cl. A61L 15/14; A61K 9/127; A61K 31/785; A61P 31/00

(52) U.S. Cl. 424/445; 424/450; 424/78.08

(57) ABSTRACT

The present invention relates to biguanide-containing liposomes and antiseptic preparations based on biguanide-containing liposomes, where the liposomes are characterised in that they are essentially free of lipids with anionic head groups, to the preparation of biguanide-containing liposomes and of the antiseptic preparations, and to their possible uses and to the products arising from their use, in particular wound dressings.

### CROSS-REFERENCE TO RELATED APPLICATIONS

This application is a Section 371 of International Application No. PCT/EP2007/002287, filed Mar. 15, 2007, which was published in the German language on Oct. 18, 2007, under International Publication No. WO 2007/115635 A1 and the disclosure of which is incorporated herein by reference.

### BACKGROUND OF THE INVENTION

The present invention relates to biguanide-containing liposomes, to antiseptic preparations based on liposomes which contain at least one biguanide as microbicidal active agent, to the preparation of the biguanide-containing liposomes and the antiseptic preparations, and to their possible uses and to the products arising from their use.

The healing process of a wound deteriorates markedly if a wound dries out, if there is a high number of bacteria present in the wound and/or if the wound is treated with toxic agents. By contrast, a moist environment, lack of colonization with pathogens and the increased presence of growth factors promote epithelialization and the formation of granulation tissues. The advantages of moist wound healing, where a moist environment in the wound is desired in order to stimulate cell growth and thus to achieve the quickest possible regeneration of the damaged tissue, are undisputed among the experts. On the other hand, a moist environment also creates good growth conditions for bacteria. Microbial contamination of wounds, however, interferes considerably with the process of wound healing and poses an ever increasing problem, especially in recent years, due to the increasing resistance of microorganisms to antibiotics.

As a low-grade colonization of wounds with microorganisms (colloquially “microbes”) is the rule, spreading of the microbial contamination of wounds should be prevented or avoided as much as possible. In particular, a contamination with multiresistant pathogenic organisms, e.g., methicillin-resistant Staphylococcus aureus (MRSA), necessitates treatment to prevent the contamination from spreading farther.

Wounds contaminated or infected with microorganisms should therefore be treated antiseptically because:

infection may develop as long as the wound is colonized with microorganisms;

the wound healing process remains incomplete, or is completed only with delay, as long as the wound remains infected;

the infection of the wound may spread and lead to sepsis; and

in the case of a wound colonization with multiresistant pathogens, the further spread of these pathogens must be prevented.

Particularly in the case of burn wounds there is a necessity of early prevention of wound infections, especially where contamination of larger wound areas is to be expected. The technical literature describes various methods for cleaning wounds having even microorganisms that are resistant to antibiotics. It has, for example, been proposed to remove plaques and coarse contamination from the wound by means of rinsing solutions. Usually, Ringer's solutions, Ringer's lactate solutions or isotonic sodium chloride solutions are utilized for that purpose. These solutions, however, do not have antiseptic activity and they merely result in a cleansing of the wound.

Solutions for cleaning wounds which have antimicrobial activity are also available on the market. These products are, for instance, iodine solutions, hydrogen peroxide solutions, silver salt solutions or polyhexanide solutions, all of which possess certain drawbacks, however.

Iodine has an aggressive oxidative effect, which leads to a reliable microbicidal activity against gram-positive and gram-negative bacteria, fungi and protozoa, as well as against a number of viruses. Providing the water-soluble povidone-iodine (PVP iodine, polyvinyl pyrrolidone iodine) enables a wound treatment which is mostly free of pain, as compared to the use of the previously commonly utilized alcohol-containing iodine solutions (iodine tincture), but many contraindications and problems still persist in the use of PVP iodine. For example, iodine from PVP iodine is taken up by the organism via the skin, iodine allergies and iodine intolerances are known which also conflict with the use of PVP iodine, and it has been demonstrated that iodine inhibits cell division. Consequently, the treatment of wounds with PVP iodine leads to delayed wound healing. By incorporating PVP iodine in liposomes, commercially available under the name Repithel® (Mundipharma GmbH, Limburg, DE), the tissue tolerance of PVP iodine could be markedly improved, without interfering with the efficacy of the PVP iodine. The base substance of Repithel® is a polyacrylate gel which, apart from water, contains so-called hydrosomes, that is, special multilayer liposomes. These liposomes, which are built up of several phospholipid bilayers arranged like onion skins, contain, apart from PVP iodine as a low-dosed antiseptic, also a large quantity of water. Repithel® is thereby able to release and absorb water like conventional hydrogel formulations. In this way, it creates a moisture balance.

However, Repithel®, just like PVP iodine as such or the well-known iodine tincture, leads to a mostly temporary coloring of the treated area. The inherent brown color of PVP iodine indicates the efficacy of the PVP iodine-containing preparation, but it also leads to staining of textiles. In addition, when applying Repithel® or any other iodine-containing preparations, the following contraindications have to be taken into consideration:

hyperthyroid diseases of the thyroid, dermatitis herpetiformis Duhring, hypersensitivity to iodine. Furthermore, Repithel® is not to be used prior to and following a radiotherapy. Also during pregnancy and lactation, as well as with newborns and infants up to the age of 6 months, iodine-containing preparations should be utilized only after extremely careful consideration and with monitoring of the thyroid gland function by a physicist.

Hydrogen peroxide in the wound quickly disintegrates to water under release of oxygen. The released oxygen can oxidise the cell walls of the contaminated bacteria. Because of its foaming activity caused by the rapid release of oxygen, particularly contaminated and/or incrusted wounds can be successfully mechanically cleaned with hydrogen peroxide. On the other hand, treatment with hydrogen peroxide also results in a superficial chemical burn of the wound, which at least protracts wound healing. Hydrogen peroxide is therefore not suitable for long-term application, especially in the case of chronic wounds.

Silver salt solutions act as a bactericide by destroying the bacteria's cell wall and denaturing the bacterial enzymes. Problems are, however, the insufficient stability of silver nitrate solutions, the possible absorption of silver ions, and the destruction of the skin's surface because of the protein coagulation caused by the silver. For these reasons, experts have for some time considered the use of silver salt solutions for the treatment of wounds to be outdated. The use of silver sulfadiazine, a complex of silver and the sulfonamide sulfadiazine, is no longer considered acceptable, if only because of the antibiotic portion.

Ready-to-apply polyhexanide solutions for wound healing are commercially available under the name Lavasept® (Fresenius AG, Bad Homburg, DE) or Prontosan® (B. Braun Petzold GmbH, Melsungen, DE). Polyhexanide (polyhexamethylene biguanide; PHMB) is considered a local antiseptic having a broad range of action and good tolerance. Via its cationic charges, polyhexanide acts antimicrobially by increasing the permeability of the bacterial cell membrane and by leading to the death of the cells via the loss of potassium and of other components of the cytoplasm caused by the increased permeability. Because of its comparatively slow onset of action, polyhexanide is recommended for repeated application to chronically poorly healing or sensitive wounds.

A disadvantage of the use of polyhexanide solutions, however, is the fact that this antiseptic loses its activity in the presence of even small amounts of negatively charged ions, e.g., in the presence of alginate, acrylate, lactate or iodide ions. For this reason, care must be taken that polyhexanide solutions are not used together with other wound therapeutics and/or modern wound bandages. Also when choosing wound coverings, one has to make sure that these are free of active agents.

Apart from the antiseptic solutions, a number of wound dressings are available which additionally contain active agents that are to protect the dressing from microbial colonization and to reduce the number of germs in the wound. Especially wound dressings that contain silver or silver salts as antimicrobial finish are widely used, for example the products Actisorb® (Johnson & Johnson W M, Norderstedt, DE) and Contreet®-H (Coloplast GmbH, Hamburg, DE). The Actisorb® wound dressing, which is a combination of elemental silver and activated charcoal is used especially for infected and exulcerating wounds to remove unpleasant smells. Contreet®-His a hydrocolloid dressing with enclosed silver ions which, depending on the exudation behaviour of the wound, produces antiseptic silver concentrations in the wound.

For a short time there have also been wound dressings based on collagen, cellulose derivates or alginates which contain polyhexanide as antimicrobial active agent in concentrations of, in most cases, 0.5 to 2%. These wound dressings are produced by spraying or impregnating the base material or the carrier material with an aqueous solution containing polyhexanide. It has turned out, however, that polyhexanide binds excellently to the base or carrier materials that are commonly used for the production of wound dressings and bandages. This interferes with the release of polyhexanide from the wound dressings and, as a consequence, with the antimicrobial activity of the polyhexanide.

### BRIEF SUMMARY OF THE INVENTION

The task underlying the present invention was to provide an antiseptic preparation by means of which wounds can be cleaned and/or treated, which has a broad range of action and good tolerance, which leads neither to denaturing phenomena nor to discoloration of the wound or of objects which during the treatment of the wound come into contact with the preparation or with the treated wound, and which is suitable also for long-term application, for example in the case of chronic wounds.

One object of the invention was to provide an antiseptic preparation by means of which the concept of moist wound treatment can be retained without having to fear a contamination of the wound or of a wound dressing that has to be used.

It was another object of the invention to indicate a method for the production of liposomes which are stable and contain at least one biguanide with antimicrobial activity.

A further object of the invention was to provide antiseptic wound dressings comprising at least one biguanide with antimicrobial activity, wherein the availability of the biguanide and, as a consequence, its antimicrobial activity, has been improved.

In accordance with the invention, these objects are achieved by providing liposomes of a specific composition which contain at least one biguanide with antimicrobial activity. Thus, it is possible to produce stable liposomes in the presence of biguanides if in the production of the liposomes no lipids are used that have an anionic head group, for example, phosphatidyl glycerol. For producing and loading liposomes with biguanides, the crossflow injection method described in WO 02/36257 has turned out to be particularly advantageous because of its very gentle process conditions and high efficiency. The production of liposomes loaded with biguanides is, however, not limited to that method. Other methods known from the prior art for the production and loading of liposomes can also be used for this purpose, for example high pressure homogenization methods, microfluidizer methods or ultrasound methods. The biguanides that may be incorporated in the liposomes according to the invention are preferably selected from the group of the pharmacologically acceptable biguanides which comprises 1,1′-hexamethylene-bis-{5-(4-chlorophenyl)-biguanide} (chlorhexidine), 1,1′-hexamethylene-bis-{5-(4-fluorophenyl)-biguanide} (fluorhexidine), polyhexamethylene biguanide (PHMB), alexidine (N,N″-bis(2-ethyl hexyl)-3,12-diimino-2,4,11,13-tetraazatetradecanediimidamine; 1,1′hexamethylene-bis[5-(2-ethylhexyl)biguanide]) and the polyhexamethylene biguanide compounds of the Vantocil® IB type (ICI). In addition, the biguanides may also be selected among the pharmacologically acceptable biguanides of the compounds described in U.S. Pat. Nos. 2,684,924; 2,990,425; 3,468,898; 4,022,834; 4,053,636; 4,198,392; 4,891,423; 5,182,101; and 6,503,952; and in GB 705,838 and GB 702,268. In particularly preferred embodiments, the biguanides are incorporated in the liposomes in the form of their water-soluble, physiologically acceptable salts. For example, polyhexamethylene biguanide hydrochloride, chlorhexidine digluconate, chlorhexidine diacetate, chlorhexidine dihydrochloride and alexidine hydrochloride are particularly preferred biguanides that may be incorporated in the liposomes.

For the production of wound dressings, the liposomes according to the invention can be used in almost any form of appearance since the antimicrobial properties of the biguanide-containing liposomes, surprisingly, persist even after the liposomes have been freeze-dried.

### DETAILED DESCRIPTION OF THE INVENTION

In the following, the invention will be described in more detail by means of especially preferred embodiments. However, these indications are not to be construed as limiting the nature of the invention to the embodiments described hereinbelow.

Surprisingly, it has been found that polyhexanide can be stably incorporated in liposomes if the liposomes are essentially free of lipids with anionic head groups.

Liposomes can be loaded with active agents either by loading the lipid layer or by loading the intraliposomal aqueous phase. As polyhexanide is a water-soluble substance, it was assumed that these molecules cannot be stably incorporated in the lipid bilayer of liposomes, but should remain in the intraliposomal phase. It was therefore attempted to load the aqueous phase with polyhexanide.

To this end, liposomes of various compositions were prepared in a suitable loading buffer, preferably Tris-HCl (pH 7.5) and HEPES (pH 7.0). To prepare the liposomes, the respective lipids used were dissolved in 96% ethanol and were injected, by pressure-controlled injection, into the aqueous phase using the crossflow injection method. The size of the liposomes thus being formed can be adjusted by means of the local lipid concentration at the injection point, which is determined by the lipid concentration in the ethanolic phase, the ethanol concentration, the injection pressure, the injection bore and the flow rate of the aqueous phase at the injection point. Immediately after injection of the lipids into the aqueous phase, the suspension was diluted with a further quantity of the aqueous phase to reduce the ethanol concentration to a tolerable level, preferably to 7.5 to 15%.

As a preliminary test, initially, 2 liposome suspensions were prepared. Suspension #1 consisted of hydrogenated soya phosphatidylcholine (S100-3=87% distearylphosphatidylcholine (DSPC) and 13% dipalmitoylphosphatidylcholine (DPPC; 10 μmol/ml)) as well as cholesterol (2 μmol/ml). Suspension #2 consisted of hydrogenated soya phosphatidylcholine (5 μmol/ml), egg phosphatidylglycerol (E-PG; 5 μmol/ml) and cholesterol (2 μmol/ml). Both liposome suspensions were examined with respect to vesicle size and size distribution by means of dynamic light scattering and—with an average vesicle size of 120 to 130 nm in diameter and a polydispersity index (PDI), serving as a measure for the scattering, of 0.23 to 0.24—yielded comparable preparation results.

Subsequently, Cosmocil® CQ (Arch Chemicals, Inc, US) was added to these liposome suspensions (9 volume parts liposome suspension+1 volume part Cosmocil® GQ), Cosmocil® GQ being a 20%, aqueous polyhexanide solution (poly(iminimidocarbonyl)iminohexamethylene hydrochloride solution), so that the liposome suspension contains 2% polyhexanide. Within a few minutes a strong sedimentation was observed in suspension #2, but not in suspension #1. Comparative light scattering measurements showed no change in the liposome size in suspension #1. In the case of suspension #2 with polyhexanide, the comparative measurement, yielding an average vesicle size of >4,000 to 5,000 nm, showed a marked change as compared to the liposome size in suspension #2 without added polyhexanide.

The preparation of liposomes with the same lipid compositions as in suspension #1 and suspension #2 in the presence of polyhexanide likewise yielded a comparable result. To prepare these liposomes, Cosmocil® GQ 1:1 was diluted with PBS to prepare a 10% polyhexanide solution. This solution was injected at 55° C. into the lipid/ethanol solution using crossflow injection methods, and subsequently diluted 1:5 with PBS. Suspension #3 (S 100-3, cholesterol and polyhexanide) contained liposomes with an average size of 360 to 370 nm, as demonstrated by the light scattering measurement. Suspension #4 (S 100-3, E-PG, cholesterol and polyhexanide), however, showed a sedimentation behavior similar to that of suspension #2 after addition of polyhexanide. The vesicle size in suspension #4 was 3,000-4,000 nm. These results show that liposomes can be prepared in the presence of polyhexanide provided that no phosphatidylglycerol is incorporated into the liposome membrane. Phosphatidylglycerol is a phospholipid with an anionic head group. It was furthermore observed in these tests that repeated slight clouding occurred in the PBS/polyhexanide solutions during the heating process. Therefore, PBS does not appear to be an optimal buffer for the preparation of polyhexanide-containing liposomes, even though PBS is, in principle, suitable as a buffer for the aqueous phase.

Instead of PBS buffer, various buffers were selected for preliminary trials, taking care that the buffer was suitable for use at about pH 7.0 since the lipids taken into consideration for the preparation of the liposomes show a good stability at that pH value. In these tests it was found that 50 mM citric acid (pH 7.0) led to a strong turbidity reaction upon adding polyhexanide, which, although disappearing when the process temperature was heated to 40 to 50° C., reoccurred upon cooling. 20 mM Tris buffer (pH 7.5) and 20 mM HEPES buffer (pH 7.0), for example, are to be mentioned as buffer solutions that turned out to be particularly suitable for diluting polyhexanide in connection with the manufacture of polyhexanide-containing liposomes; both did not lead to a turbidity reaction in the presence of polyhexanide. In addition, the use of these two last-mentioned buffers yielded polyhexanide-containing liposomes that were not distinguishable in terms of their size, loading with polyhexanide, and stability.

In another series of tests, different lipids were used for producing polyhexanide-containing liposomes, namely
S 100-3=hydrogenated soya phosphatidylcholine (mixed-chain, hydrogenated phospholipid of 87% distearylphosphatidylcholine (DSPC) and 13% dipalmitoylphosphatidylcholine (DPPC));
E-PC=phosphatidylcholine from egg (natural, mixed-chain phospholipid with unsaturated fatty acids);
E80 S=mixture of 80% phosphatidylcholine (E-PC) and 20% phosphatidylethanolamine (E-PE; 1-palmitoyl-2-oleoyl-sn-glycero-3-phosphoethanolamine) from egg (natural, mixed-chain phospholipid with unsaturated fatty acids and a charged moiety);
DMPC=dimyristoylphosphatidylcholine; and
DPPC=dipalmitoylphosphatidylcholine.

The size distribution of the liposomes yielded a heterogeneous result. Liposomes produced with S 100-3 at 55° C. with 10% polyhexanide showed, in the absence of cholesterol (suspension #6), a monomodal distribution with an average liposome size of 200-250 nm. By contrast, the presence of cholesterol (suspension #5) resulted in a multimodally distributed liposome suspension, which was not used for further examinations. Polyhexanide-containing liposomes which had been produced with E-PC at 35° C. yielded, in the presence of cholesterol (suspension #7), a homogenous monomodal vesicle population with a diameter of 200 nm on average, but not in the absence of cholesterol (suspension #8). With suspensions that were produced with E 80-S, the presence or absence of 15 to 20 mol % cholesterol (suspensions #9 and #10) had no influence on the formation of liposomes. Cholesterol is even likely to be applicable in quantities of up to or slightly below 50 mol % without interfering with liposome formation since cholesterol is not a membrane-forming lipid.

The monomodal size distribution of the liposomes in suspensions #6, #7, #9 and #10 lead to the conclusion that there is little or no interaction between polyhexanide and the membrane lipids. In the case of the multimodal size distributions, the polyhexanide presumably interacts with the membrane or with individual components thereof and thus disturbs a homogenous vesicle formation. Only liposome suspensions with a monomodal size distribution of the vesicles were used for further analyses and preparations. These suspensions were filtrated in a 10 ml Amicon stir cell with a cellulose acetate membrane, which had a size exclusion limit of 100 kDa, to remove the portion of unincorporated polyhexanide. Subsequently, the polyhexanide content of all the fractions obtained in the filtration was determined using the eosin test method. On average, 15 to 25% of the added polyhexanide had been incorporated in the liposomes examined. Analysis of the filtrates also showed that polyhexanide remains stable in the liposomes, that the amount of polyhexanide in the successive filtrates decreased continuously, and that the polyhexanide content in the retentate was within the expected range.

Corresponding tests were also carried out with the synthetic lipids DPPC (dipalmitoylphosphatidylcholine) and DMPC (dimyristoylphosphatidylcholine). In these tests it was found that with DPPC, the liposome suspensions prepared at 50 to 55° C. exhibited a multimodal size distribution, independent of the presence of cholesterol (suspension #11) or the absence of cholesterol (suspension #12). Liposomes from DMPC and cholesterol (suspension #13) which, like the liposomes from the unsaturated lipid mixtures (suspensions #7 and #8), had been prepared at 35° C. showed better results, namely an average diameter of the liposomes of about 400-500 nm and a stable incorporation of about 30-40% of the added PHMB.

Since it could not be clarified beyond doubt whether the Tris buffer that had been used for the aforementioned tests can be used for pharmaceutical formulations or medical products, the liposome preparations which when using Tris buffer had yielded a satisfactory result, namely suspensions #7, #9, #10 and #13, were repeated using 20 mM HEPES buffer (pH 7.0) instead of Tris buffer, since HEPES buffers, as has been demonstrated, are being utilized in pharmaceutical formulations.

No differences were found between the liposomes prepared with Tris buffer and those prepared with HEPES buffer, as is shown by the comparison of vesicle sizes and polyhexanide loading of suspensions #7, #9, #10 and #13 with those of suspensions #15 to #18 (see Table 1). Both with respect to the size of the vesicles and their size distribution, and in the encapsulating behavior of polyhexanide, no significant differences were found. Only the E-PC/cholesterol-containing liposome suspensions (suspensions #7 and #15) showed a wider spread of the measured values in the analyses than the remaining liposome preparations.

In further experiments, vitamin E was additionally added to the lipid/ethanol solution in the preparation process in order to protect the liposomes prepared from unsaturated lipids from oxidative degradation during their storage and to improve wound healing by adding this radical scavenger.

First, the optimum amount of vitamin E not adversely affecting the vesicle formulation process was determined. As a result, it was found that up to 40 mol % of vitamin E could be added to the lipid phase without interfering with the vesicle formation. On the basis of these results, polyhexanide-containing liposomes with 20 mol % vitamin E were prepared in a further series, and subsequently filtrated and analyzed. The properties of the liposomes produced from these preparations (suspensions #19 to #21) were identical, in terms of vesicle size and encapsulated amounts of polyhexanide, to the properties of the liposomes from the preparations without addition of vitamin E (suspensions #15, #16, #17). When using vitamin E, about 20 to 25% of the added PHMB was likewise liposomally incorporated. The loading of liposomes with polyhexanide is dependent on their vesicle size. Liposomes with a diameter of 150 to 200 nm can be loaded constantly with 15 to 20% of the polyhexanide added to the preparation, liposomes with a diameter of 400 to 500 nm can be loaded constantly with 30 to 40% of the polyhexanide added to the preparation. To prepare antiseptically active liposome suspensions, apart from the preferred polyhexanide, whose good tolerance and wide range of action has been proved, it is also possible to use any other biguanides that possess antimicrobial activity and are physiologically acceptable. The molecular weights of the polyhexanides to be used are not subject to any substantial limitations. Polyhexanides of any molecular weights as have been usually used to date, may be used. The preferred PHMB has a molecular weight in the range of from 1,500 to 15,000 g/mol. Preferred polyhexanides are those having a degree of polymerization of 12-16. The degree of polymerization indicates the average number of monomer molecules that are connected to form one macromolecule during the polymerization.

Incorporation of liposomally incorporated polyhexanide or of another liposomally incorporated biguanide into a wound dressing can be accomplished in different ways. The polyhexanide-containing liposomes may, for example, be incorporated in a polymer solution of the carrier material for the wound dressing. The solvent is subsequently withdrawn by evaporation or freeze drying. To produce a moist wound dressing, the solvent may completely or in part remain in the wound dressing, respectively in the carrier material for the wound dressing, before the latter is processed further.

As an alternative, the suspension of biguanide-containing liposomes may also be applied to the carrier material using methods which are employed with polyhexanide solutions. Thus, the biguanide-containing liposomes may be applied to the carrier material by sprinkling or spraying.

The present invention thus relates to antiseptic preparations which are based on an active agent enclosed in liposomes and which are characterized in that the liposomes do not contain lipids with anionic head groups, have an aqueous medium in their interior, and in that at least one antimicrobial active agent from the group of the biguanides is contained in the aqueous medium.

TABLE 1
Overview of the compositions and properties
of the liposome suspensions prepared

| | | | | Size | |
|---|---|---|---|---|---|
| | | | | distribution | Loading |
| | | Aqueous | Size of the | of the | with |
| # | Lipid phase | Phase | vesicles | vesicles | PHMB |
| 1 | S 100-3 | PBS | 120-130 | monomodal | |
| | Cholesterol | | | | |
| 2 | S 100-3 | PBS | >5000 | monomodal | |
| | Cholesterol | | | | |
| | E-PG | | | | |
| 3 | S 100-3 | PHMB | 360-370 | bimodal | |
| | Cholesterol | PBS | | | |
| 4 | S 100-3 | PHMB | >5000 | monomodal | |
| | Cholesterol | PBS | | | |
| | E-PG | | | | |
| 5 | S 100-3 | PHMB | | multimodal | |
| | Cholesterol | Tris | | | |
| 6 | S 100-3 | PHMB | 200-250 | monomodal | 15-25 |
| | | Tris | | | |
| 7 | E-PC | PHMB | 200 | monomodal | 15-25 |
| | Cholesterol | Tris | | | |
| 8 | E-PC | PHMB | | multimodal | |
| | | Tris | | | |
| 9 | E 80-S | PHMB | 150-200 | monomodal | 15-25 |
| | Cholesterol | Tris | | | |
| 10 | E 80-S | PHMB | 150-200 | monomodal | 15-25 |
| | | Tris | | | |
| 11 | DPPC | PHMB | | multimodal | |
| | Cholesterol | Tris | | | |
| 12 | DPPC | PHMB | | multimodal | |
| | | Tris | | | |
| 13 | DMPC | PHMB | 400-500 | monomodal | 30-40 |
| | Cholesterol | Tris | | | |
| 14 | DMPC | PHMB | | | |
| | | Tris | | | |
| 15 | E-PC | PHMB | 200 | monomodal | 15-25 |
| | Cholesterol | HEPES | | | |
| 16 | E 80-S | PHMB | 150-200 | monomodal | 15-25 |
| | Cholesterol | HEPES | | | |
| 17 | E 80-S | PHMB | 150-200 | monomodal | 15-25 |
| | | HEPES | | | |
| 18 | DMPC | PHMB | 400-500 | monomodal | 30-40 |
| | Cholesterol | HEPES | | | |
| 19 | E-PC | PHMB | 200 nm | monomodal | 15-25% |
| | Cholesterol | HEPES | | | |
| | Vitamin E | | | | |
| 20 | E80-S | PHMB | 150-200 m | monomodal | 15-20% |
| | Cholesterol | HEPES | | | |
| | Vitamin E | | | | |
| 21 | E 80-S | PHMB | 150-200 nm | monomodal | 15-25% |
| | Vitamin E | HEPES | | | |
| 22 | DMPC | PHMB | 400-500 nm | monomodal | 30-40% |
| | Vitamin E | HEPES | | | |

Preferably, the liposomes according to the present invention include phospholipids selected from the group of the natural and synthetic phospholipids which comprises phosphatidylcholine, phosphatidylethanolamine, dimyristoylphosphatidylcholine and mixtures thereof. The natural phospholipids preferably originate from eggs or soya beans.

If synthetic phospholipids are used to produce the liposomes, it is possible to use phospholipids with chain lengths of 14 to 24 carbon atoms. The longer the carbon chains of the phospholipids, the better the biguanide is likely to remain in the liposomes. In a particularly preferred embodiment, the composition according to the invention comprises polyhexanide as linear polymer biguanide with antimicrobial activity, with polyhexanide of a molecular weight of 1,500 to 15,000 g/mol and/or with a degree of polymerization of 12-16 being especially preferred.

The aqueous medium preferably is a buffer, with PBS, Tris buffer and HEPES buffer being especially preferred. The aqueous medium should have a pH value of 6 to 8; preferably the pH value is 7.0 to 7.5.

In a preferred embodiment the liposomes, more precisely the lipid bilayers of the liposomes, contain cholesterol.

The cholesterol content in the liposomes can amount to up to 50 mol %; preferably, the cholesterol content is 15 to 20 mol %.

In another preferred embodiment, the liposomes, more precisely the lipid bilayers of the liposomes, contain vitamin E in an amount of up to 40 mol %, preferably in an amount of 20 mol %, in each case relative to the total lipids, where applicable in addition to the cholesterol.

The liposomes according to the invention preferably have a mean size of 50 to 800 nm; liposomes having a means size of 150 to 500 nm are especially preferred.

The preparation according to the invention may, for example, be present in the form of a suspension, emulsion, lotion, tincture, a spray, gel, a cream or an ointment.

The present invention also relates to methods for producing antiseptic compositions based on an antimicrobial active agent from the group of the biguanides which is enclosed in liposomes, wherein the liposomes are free of lipids with anionic head groups and where the method is characterized in that initially an ethanolic lipid phase is injected, by pressure-controlled injection, into an aqueous phase containing the antimicrobial active agent from the group of the biguanides, that after the formation of vesicles has taken place, the aqueous phase is diluted with a buffer, and that unincorporated active agent is subsequently removed. Preferably, the buffer used for diluting the liposome suspension is the same as that used for preparing the aqueous phase.

In the method according to the invention, the phospholipids used for the lipid phase are preferably phospholipids from the group of the natural or synthetic phospholipids which are selected from the group which consists of phosphatidylcholine, phosphatidylethanolamine, dimyristoylphosphatidylcholine and mixtures thereof, with the natural phospholipids preferably originating from eggs or soya beans.

Preferably, chlorhexidine, fluorhexidine, alexidine or polyhexanide are used as the biguanide with antimicrobial activity; especially preferred are polyhexanides with a molecular weight of 1,500 to 15,000 g/mol and/or with a degree of polymerization of 12 to 16.

In the method according to the invention, the aqueous phase is preferably prepared from a buffer system, especially preferably from the group consisting of PBS (phosphate-buffered saline), Tris buffer and HEPES buffer.

The pH of the aqueous phase is adjusted to a value of from 6 to 8; especially preferably, the pH value is 7.0 to 7.5. In a particularly preferred method, the lipid phase contains cholesterol in an amount of 0 to 50 mol %, preferably of 15 to 20 mol %, and/or vitamin E in an amount of 0 to 40 mol %, preferably 20 mol %, in each case relative to the total lipids.

The present invention also relates to the use of the antiseptic composition according to the invention, more particularly the use thereof for producing wound dressings the carrier material of which can be provided with the antimicrobial liposomes, for example by sprinkling, spraying or impregnating.

Suitable as the carrier materials for the production of the wound dressings are any materials which are conventionally used for this purpose and known to those skilled in the art, for example collagen, celluloses and cellulose derivatives, polyurethane, alginates, alone or in combination with polysaccharides from the group which consists of alginates, hyaluronic acid and its salts (hyaluronates), pectins, carrageenans, xanthans, sulfated dextrans, cellulose derivatives, oxidized cellulose such as oxidized regenerated cellulose, chondroitin, chondroitin-4-sulfate, chondroitin-6-sulfate, heparin, heparan sulfate, keratan sulfate, dermatan sulfate, starch derivatives, and mixtures thereof.

In another preferred use, a polymer solution, for example a collagen solution, is mixed with the liposome suspension, and the solvent or solvents is/are subsequently, completely or partially, removed by drying or freeze drying so that sponges can be obtained which are provided with the antimicrobially active liposomes.

The present invention thus also relates to wound dressings which comprise biguanide-containing liposomes and are based on, for example, cellulose, a cellulose derivative, such as carboxymethyl celluloses, alginates, chitosan, starch or starch derivatives, collagen, polyacrylates, polyurethane, or mixtures of the aforementioned compounds as carrier material.

The preferred embodiments of the wound dressings according to the invention are hydrogels, hydrocolloids, sponges, films, membranes, nonwoven fabrics, woven fabrics, knit fabrics, other textile fabrics, card slivers, tamponades, and the like. Especially preferably, the wound dressings according to the invention contain the liposomally incorporated, antimicrobially active biguanide, preferably polyhexanide, in an amount of 0.01 to 1.0 wt %., relative to the dry weight of the dressing.

### Embodiment Example

To prepare collagen sponges with an antiseptic finish, 1% collagen suspensions (of bovine origin) were thoroughly mixed with defined amounts of a liposome suspension (in Tris buffer), and subsequently placed in a plastic dish. The mixture was then deep-frozen at −50° C. and subsequently lyophilized. In this way, collagen sponges with a polyhexanide content of 0.05 wt %., 0.1 wt %, 0.5 wt % or 1 wt %., relative to the dry weight of the collagen, were prepared. Three different liposome suspensions were used:
I. liposomes of E 80-S, cholesterol and vitamin E (suspension #20);
II. liposomes of E 80-S and vitamin E (suspension #21); and
III. liposomes of DMPC and cholesterol (suspension #22).

The antimicrobial activity of the collagen sponges comprising polyhexanide-containing liposomes and of the liposome suspensions used for producing these collagen sponges on Staphylococcus aureus, Pseudomonas aeruginosa and Candida albicans was examined in more detail by means of agar diffusion tests. To this end, the microorganisms were cultivated for 24 h in a non-selective liquid medium at 30 to 35° C. and were subsequently diluted with a 1% NaCl solution containing 1% peptone to 1×108 CFU/ml (colony forming units) and 3.8×107 CFU/ml (C. albicans), respectively. 100 μl of each of the dilutions were spread onto CSA and SDA plates, respectively (CSA=casein-soya-peptone agar; SDA=Sabouraud-dextrose agar). The agar plates were dried for 3 to 5 minutes.

After applying the solutions, liposome suspensions or collagen sponges, the plates were incubated for 24 h at 30 to 35° C. and for 48 h at 20 to 25° C. (C. albicans), respectively, before determining the region of inhibition. The area of inhibition was quantified by measuring the distance from the sponge, or from the hole in the agar plate for the solution or suspension to be filled in, to the edge of the inhibiting areola. The results are summarized in semiquantitative form in Table 2. The collagen sponges comprising liposomes loaded with polyhexanide showed good antimicrobial activity towards the three examined microorganisms (S. aureus, P. aeruginosa and C. albicans). In these examinations, the collagen sponges with polyhexanide-containing liposomes based on DMPC showed the weakest microbicidal activity towards the examined microorganisms, as compared to the collagen sponges which had been loaded with polyhexanide-containing liposomes based on E 80-S. The collagen dressings with polyhexanide-containing liposomes based on E 80-S showed good activity towards S. aureus and C. albicans, even if loaded with only small amounts of polyhexanide. The microbicidal activity of these wound dressings towards P. aeruginosa was inconsistent; however, even in the case of the wound dressings with a low polyhexanide loading, no microbial contamination of the wound dressing occurred. Freeze drying of the liposome suspensions had no negative effects on the antimicrobial activity of the preparations.

The present results show that by using liposomally incorporated polyhexanide, it is possible to produce wound dressings, at least on the basis of collagen or with collagen, that have excellent antimicrobial activity.

TABLE 2
Semiquantitative evaluation of the antimicrobial activity of polyhexanide-containing preparations

| | Composition | Type of preparation | PHMB content | S. aureus | P. aeruginosa | C. albicans |
|---|---|---|---|---|---|---|
| A | Liposomes of | collagen sponge | 1.0 | +++ | + | +++ |
| | E 80-S | | 0.5 | +++ | + | +++ |
| | Cholesterol | | 0.1 | ++ | + | +++ |
| | Vitamin E | | 0.05 | ++ | + | +++ |
| B | Liposomes of | collagen sponge | 1.0 | +++ | +++ | +++ |
| | E 80-S | | 0.5 | +++ | +++ | +++ |
| | Vitamin E | | 0.1 | +++ | + | +++ |
| | | | 0.05 | +++ | + | ++ |
| C | Liposomes of | collagen sponge | 1.0 | ∘ | ∘ | ∘ |
| | DMPC | | 0.5 | ∘ | ∘ | ∘ |
| | Cholesterol | | 0.1 | ∘ | ∘ | ∘ |
| | | | 0.05 | ∘ | ∘ | ∘ |
| D | Liposomes of | suspension | 1.0 | +++ | + | +++ |
| | E 80-S | | 0.5 | +++ | + | +++ |
| | Cholesterol | | 0.1 | ++ | + | +++ |
| | Vitamin E | | 0.05 | ++ | + | + |
| E | Liposomes of | suspension | 1.0 | +++ | + | +++ |
| | E 80-S | | 0.5 | +++ | + | +++ |
| | Vitamin E | | 0.1 | +++ | + | +++ |
| | | | 0.05 | +++ | + | + |
| F | Liposomes of | suspension | 1.0 | +++ | + | + |
| | DMPC | | 0.5 | +++ | + | + |
| | Cholesterol | | 0.1 | +++ | + | + |
| | | | 0.05 | +++ | + | + |
| H | Solution of | collagen sponge | 1.0 | +++ | + | + |
| | PHMB | | 0.5 | +++ | + | + |
| | | | 0.1 | ++ | + | ∘ |
| | | | 0.05 | ++ | + | ∘ |
| I | Liposomes of | suspension. lyophilisiert | 1.0 | +++ | +++ | +++ |
| | E 80-S | | 0.5 | +++ | +++ | +++ |
| | Vitamin E | | 0.1 | +++ | ++ | +++ |
| | | | 0.05 | +++ | + | +++ |
| J | Liposomes of | suspension | 1.0 | +++ | ++ | +++ |
| | E 80-S | lyophilised | 0.5 | +++ | ++ | +++ |
| | Cholesterol | | 0.1 | +++ | + | +++ |
| | Vitamin E | | 0.05 | +++ | + | +++ |
| K | Liposomes of | suspension | 1.0 | +++ | + | + |
| | DMPC | lyophilised | 0.5 | +++ | + | + |
| | Vitamin E | | 0.1 | ++ | + | + |
| | | | 0.05 | + | + | + |
| L | PHMB | solution | 1.0 | +++ | +++ | +++ |
| | | | 0.5 | +++ | +++ | +++ |
| | | | 0.1 | +++ | +++ | +++ |
| | | | 0.05 | +++ | +++ | +++ |
| M | | collagen sponge | 0.0 | − | − | − |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-29. (canceled)

30. An antiseptic composition comprising an antimicrobial active agent enclosed in liposomes, wherein the liposomes are free of lipids with anionic head groups, and the liposomes have an aqueous medium in an interior which contains polyhexanide as the antimicrobial active agent.

31. The antiseptic composition according to claim 30, wherein the liposomes contain at least one natural or synthetic phospholipid.

32. The antiseptic composition according to claim 31, wherein the at least one natural or synthetic phospholipid has an acyl chain length of at least 14 carbon atoms.

33. The antiseptic composition according to claim 31, wherein the natural phospholipid originates from eggs or soya beans.

34. The antiseptic composition according to claim 30, wherein the polyhexanide has a molecular weight of 1,500 to 15,000 g/mol and/or a degree of polymerization of 12 to 16.

35. The antiseptic composition according to claim 30, wherein the aqueous medium comprises a buffer which is at least one selected from the group consisting of PBS, Tris buffer and HEPES buffer.

36. The antiseptic composition according claim 30, wherein a pH value of the aqueous medium is from 6 to 8.

37. The antiseptic composition according to claim 30, wherein the liposomes contain cholesterol.

38. The antiseptic composition according to claim 30, wherein the liposomes contain vitamin E.

39. The antiseptic composition according to claim 30, wherein the liposomes have a mean size of from 50 to 800 nm.

40. The antiseptic composition according to claim 30, wherein the composition is in a form of a suspension, emulsion, lotion, tincture, spray, gel, cream or ointment.

41. A method for production of an antiseptic composition including an antimicrobial active agent enclosed in liposomes, wherein the liposomes are free of lipids with anionic head groups and the liposomes have an aqueous medium in an interior which contains polyhexanide as the antimicrobial active agent, comprising:
injecting an ethanolic lipid phase, by pressure-controlled injection, into an aqueous phase containing the polyhexanide to thereby form vesicles,
diluting the aqueous phase with a buffer, and
removing polyhexanide which has not been incorporated into the vesicles.

42. The method according to claim 41, wherein the lipid phase comprises natural or synthetic phospholipids.

43. The method according to claim 42, wherein the phospholipids have an acyl chain length of at least 14 carbon atoms.

44. The method according to claim 42, wherein the natural phospholipids originate from eggs or soya beans.

45. The method according to claim 41, wherein the polyhexanide has a molecular weight of 1,500 to 15,000 g/mol and/or a degree of polymerization of 12 to 16.

46. The method according to claim 41, wherein the aqueous medium comprises a buffer which is at least one selected from the group consisting of PBS, Tris buffer and HEPES buffer.

47. The method according to claim 41, wherein a pH value of the aqueous medium is from 6 to 8.

48. The method according to claim 41, wherein the lipid phase contains cholesterol.

49. The method according to claim 41, wherein the lipid phase contains vitamin E.

50. A method of producing a wound dressing, comprising producing the wound dressing including the antiseptic composition according to claim 30.

51. The method according to claim 50, wherein a carrier material of the wound dressing is sprinkled, sprayed or impregnated with the composition.

52. The method according to claim 51, further comprising mixing a polymer solution with the composition and partially or completely withdrawing solvent by drying or freeze drying.

53. A wound dressing comprising the antiseptic composition according to claim 30.

54. The wound dressing according to claim 53, wherein the wound dressing comprises cellulose or cellulose derivatives as carrier material.

55. The wound dressing according to claim 53, wherein the wound dressing is in a form of a hydrogel, a hydrocolloid, a sponge, a film, a membrane, a nonwoven fabric, a woven fabric, a knit fabric, card slivers, or a tamponade.

56. The wound dressing according to claim 53, wherein the wound dressing contains polyhexanide in an amount of from 0.01 to 1.0 wt %., relative to the dry weight of the dressing.
